(19) United States
(12) Patent Application Publication
(10) Pub. No.: US 2025/0020658 A1
(43) Pub. Date: Jan. 16, 2025

(54) METHODS FOR SYNTHESIZING AND IDENTIFYING SYNTHETIC PROTEINS INCLUDING TARGETED THERAPEUTICS FOR NEURODEGENERATIVE DISORDERS BASED ON BIO-MIMICRY APPROACHES

(71) Applicant: University of Denver, Denver, CO (US)

(72) Inventors: Sunil Kumar, Denver, CO (US); Nicholas Stillman, Thornton, CO (US); Ryan Dohoney, Thornton, CO (US)

(73) Assignee: University of Denver, Denver, CO (US)

(21) Appl. No.: 18/770,559

(22) Filed: Jul. 11, 2024

Related U.S. Application Data
(60) Provisional application No. 63/526,255, filed on Jul. 12, 2023.

(51) Int. Cl. G01N 33/68; C40B 30/04

(52) U.S. Cl. CPC G01N 33/6845; C40B 30/04

(57) ABSTRACT

A method for synthesizing a synthetic molecule includes selecting a target protein, selecting a common precursor, providing first and second side-chains, forming a first monomer, including the common precursor and first side-chain, forming a second monomer, including the common precursor and second side-chain, evaluating the first and second monomers using an aggregation assay with the target protein to generate first results, and selecting one of the first and second monomers as a selected monomer based on the first results. Further, the method includes providing third and fourth monomers compatible with the selected monomer, forming a first dimer, including the selected monomer and third monomer, forming a second dimer, including the selected monomer and fourth monomer, evaluating the first and second dimers with the aggregation assay to generate second results, and selecting one of the first and second dimers as a selected dimer based on the second results.

### REFERENCE TO RELATED APPLICATIONS

The present application claims the benefit of U.S. Provisional Pat. App. No. 63/526,255, filed 2023 Jul. 12 and titled “Methods for Synthesizing and Identifying Synthetic Proteins including Targeted Therapeutics for Neurodegenerative Disorders Based on Bio-Mimicry Approaches,” which application is incorporated hereby in its entirety by reference.

### FIELD OF THE INVENTION

The present invention relates to therapeutics for neurodegenerative disorders. In particular, but not by way of limitation, the present invention relates to compounds suitable for use as therapeutics for neurodegenerative disorders involving undesirable protein aggregation.

### DESCRIPTION OF RELATED ART

Various pharmaceutical companies that are working in the field of therapeutics for neurodegenerative disorders, such as Alzheimer's disease and Parkinson's disease (PD), involving impairment and/or death of cells of the central nervous system. For example, PD is a progressive neurodegenerative disorder for which there is no successful prevention or intervention. The pathological hallmark for PD involves the self-assembly of functional Alpha-Synuclein (αS), which is a neuronal protein expressed at high levels in dopaminergic (DA) neurons in the brain, into non-functional amyloid structures. These amyloid structures are known to impair the regulation of synaptic vesicle trafficking, recycling, and neurotransmitter release. The process of αS aggregation impairs the function of DA neurons by formation of pathological αS oligomers and αS fibers (aggregates). For example, the pathological events in PD include αS aggregation and the spread of pathogenic αS fibers from neuron-to-neuron (via prion-like spread) and template the soluble αS into insoluble αS fibers. These processes of αS aggregation are also associated with other neurodegenerative disease, including dementia with Lewy body, Parkinson disease dementia, and multiple system atrophy.

The physiological and pathophysiological mechanisms of this impairment are illustrated in FIGS. 1A and 1B, respectively. FIG. 1A shows a physiological model of neurons with an expanded view of a portion of adjacent neurotransmitter and receptor pair, showing the expression of αS in dopaminergic neurons, where it is localized on dopamine-filled synaptic vesicles and help in dopamine release. As shown in FIG. 1B, illustrating a pathophysiological model of the blockage of the release of dopamine as a result of αS aggregation, leading to cell death.

One model of such protein aggregation is illustrated in FIG. 2. As shown in FIG. 2, a monomer may cluster to form a small oligomer. The oligomerization may involve, for example, the adhesion of monomers in close proximity due to the introduction of “hot spots” that act like glue between monomers. A sufficient aggregation of monomers may turn into a plurality of large oligomers, which in turn may polymerize to form protofibrils. The profibrils may cluster together to form amyloid fibers, which are known to be associated with neurodegenerative diseases.

Modulation of aggregation and the prion-like spread of αS is considered to be a promising potential therapeutic intervention for PD. However, the bottleneck towards achieving this goal is the identification of αS domains/sequences that are essential for aggregation.

There are more than 10 million people worldwide living with PD and the number is expected to double in 2030 and there is no identified therapy to cure or slow the progression of PD9-13. Therefore, there is a need for an improved mechanistic and therapeutic insights into as aggregation and its role in mediating PD phenotypes, which will pave the way for effective treatments for PD.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

In particular, the present disclosure describes various embodiments of methods for the synthesis and design of ligands, such as for applications against diseases involving neurodegeneration.

In an embodiment, a method for synthesizing a synthetic molecule includes selecting a target protein, selecting a common precursor, providing at least first and second side-chains compatible with the common precursor, forming a first monomer, including the common precursor with the first side-chain attached thereto, and forming a second monomer, including the common precursor with the second side-chain attached thereto. The method further includes evaluating the first and second monomers using an aggregation assay in the presence of the target protein to generate first results, and selecting one of the first and second monomers as a selected monomer based at least in part the first results. The method further includes providing at least third and fourth monomers compatible with the selected monomer, forming a first dimer, including the selected monomer with the third monomer attached thereto, and forming a second dimer, including the selected monomer with the fourth monomer attached thereto. The method further includes evaluating the first and second dimers with the aggregation assay to generate second results, and selecting one of the first and second dimers as a selected dimer based at least in part on the second results.

In embodiments, the method further includes providing at least fifth and sixth monomers compatible with the selected dimer, forming a first trimer, including the selected dimer with the fifth monomer attached thereto, and forming a second trimer, including the selected dimer with the sixth monomer attached thereto. The method further includes evaluating the first and second trimers with the aggregation assay to generate third results, and selecting one of the first and second trimers as a selected trimer based at least in part on the third results.

In embodiments, aggregation assay does not include a chromatography assay.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF DRAWINGS

FIGS. 1A illustrates a physiological model of neurons, including an inset showing an expanded view of a portion of an adjacent neurotransmitter and receptor pair.

FIG. 1B illustrates a pathophysiological model of the blockage of the release of dopamine between an adjacent neurotransmitter and receptor pair as a result of αs aggregation.

FIG. 2 shows an exemplary model of protein aggregation.

FIG. 3 shows an exemplary illustration of an αS oligomer associated with PD, including an inset showing further detail of a portion of the αS oligomer as compared with a synthetic protein mimetic, in accordance with an embodiment.

FIG. 4 shows an isolated view of the chemical structure of a portion of a variant of SK-129, in accordance with an embodiment.

FIG. 5A shows a top view of an oligoquinoline (OQ) scaffolding framework or structure, in accordance with the embodiment.

FIG. 5B shows a side view of the OQ scaffolding structure, illustrating the helical nature of the OQ scaffolding, in accordance with an embodiment.

FIG. 6, including insets, illustrates the physiological mechanisms involved in the transmission of dopamine between neurons in a healthy brain and in a brain affected by Parkinson's Disease and the rescue of the physiological role of neurons by introduction of the synthetic foldamer, in accordance with an embodiment.

FIG. 7 shows a detailed view of an OQ structure suitable for use as the basis of a scaffolding structure, in accordance with certain embodiments.

FIG. 8 shows a scaffolding structure formed of multiple units of OQ, in accordance with embodiments.

FIG. 9 shows exemplary options for side chains attachable to the scaffolding structure of FIG. 8, in accordance with certain embodiments.

FIG. 10 shows a chemical diagram of NS-163, suitable for use as a ligand in certain embodiments.

FIG. 11 shows an illustration of possible binding sites of SK129, suitable for use with certain embodiments.

FIG. 12 shows an illustration of possible binding sites of NS163, suitable for use with certain embodiments.

FIG. 13 shows a fluorescent analog of SK-129, suitable for use with certain embodiments.

FIG. 14 shows a process for using a synthetic protein molecule, in accordance with embodiments.

FIG. 15 illustrates a process for synthesizing a protein molecule using existing methods.

FIG. 16 shows a variety of options for side chains that may be required for evaluation, which would be cumbersome to do using the old method shown in FIG. 15.

FIG. 17 illustrates an improved process for synthesizing and evaluating a variety of molecules, in accordance with embodiments.

FIG. 18 illustrates a typical approach for evaluating a fragment library.

FIG. 19 shows a graphic summary of a 2-dimensional Fragment-Assisted Structure-based Technique (2D FAST), in accordance with embodiments.

FIG. 20 illustrates an exemplary 2D FAST process, in accordance with certain embodiments.

FIG. 21 is a graph comparing the relative ThT intensities of various molecules synthesized using the 2D FAST process, in accordance with embodiments.

FIG. 22 is a graph comparing the permeability of various synthetic molecules synthesized and tested using the 2D FAST approach.

FIG. 23 shows a chemical diagram of NS132, suitable for use with certain embodiments.

FIG. 24 shows a chemical diagram of NS163, suitable for use with certain embodiments.

FIG. 25 is a graph comparing the relative ThT intensities of various molecules synthesized using the 2D FAST process in the presence of αS, in accordance with embodiments. FIG. 26 illustrates an exemplary process of the synthesis of the NS132 molecule using the 2D FAST approach, in accordance with embodiments.

FIG. 27 illustrates formation of the NS163 molecule, in accordance with embodiments.

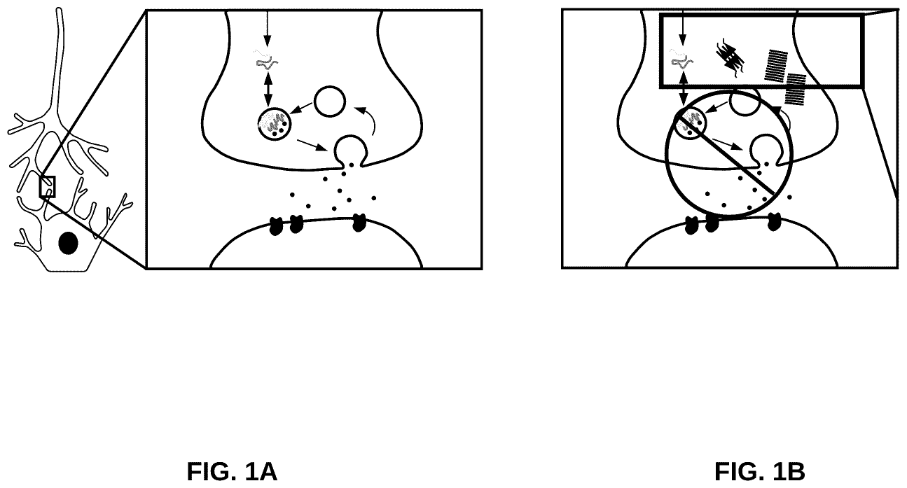

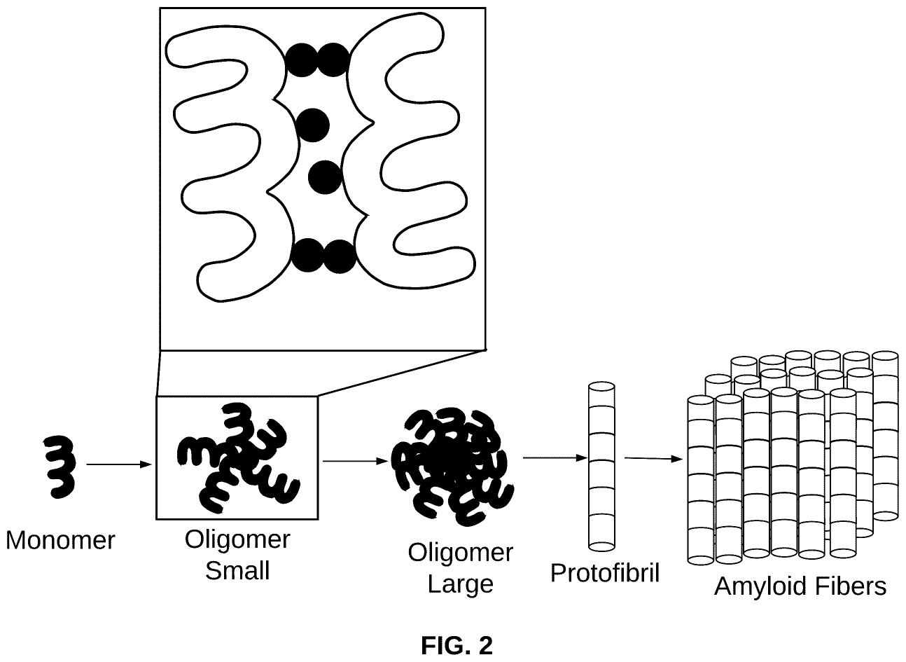

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION OF THE INVENTION

It would be desirable to identify and synthesize molecules that inhibit the formation of amyloid fiber structures by, for example, specifically binding to intermediate toxic oligomer structures of αS. Further, it would be desirable to produce such molecules that may be used in therapeutic approaches to deliver these therapeutic molecules through the blood-brain barrier.

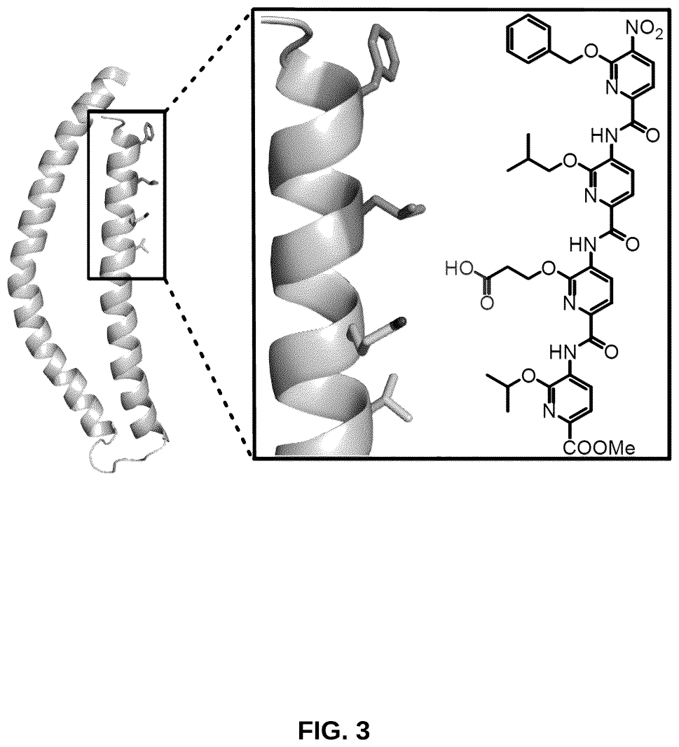

It is recognized herein that synthetic molecules that mimic the molecular features of naturally occurring molecules may be able to exhibit, in embodiments, higher affinity to specific target molecules that are similar in shape to those synthetic molecules. As a specific example, the αS oligomers associated with PD are by nature helical, as illustrated in the left-hand side of FIG. 3. These αS oligomers are considered to be highly neurotoxic structures and considered to be the main causal agents for the PD. It is further recognized herein that, in general, a helical ligand with the appropriate side-chains may attach itself to the αS structure so as to inhibit the formation of amyloid fibers. Additionally, in mimicking the helical nature of naturally occurring αS oligomers, which are neurotoxic, such helical structures are uniquely suited for being deliverable through the blood-brain barrier to the cells of the central nervous system.

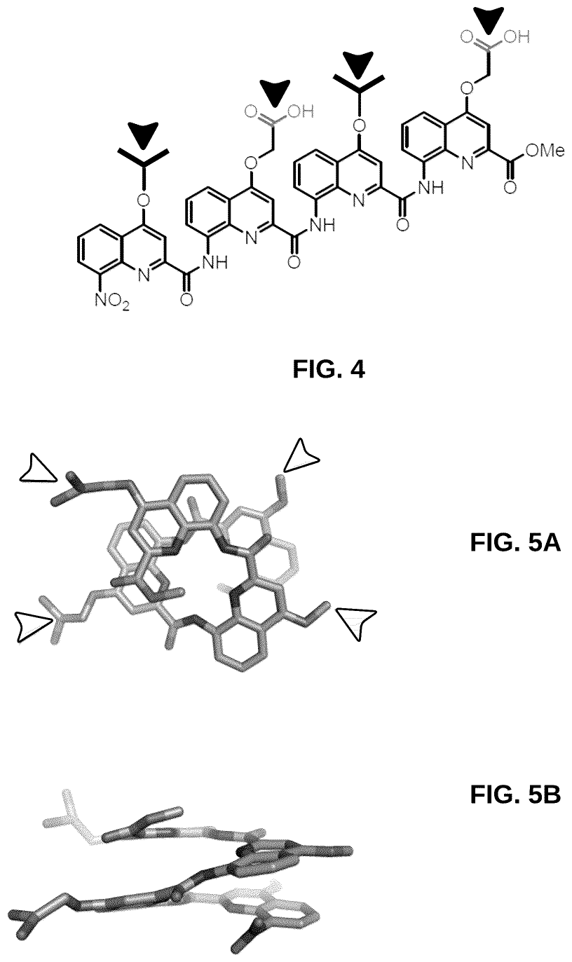

Certain specific molecules based on foldamer scaffolding, such as SK-129 based on an oligoquinoline (OQ) scaffolding have been previously studied for their potential effects on disrupting self-assembly of αS oligomers into non-functional amyloid structures (see, for example, Ahmed, et al., “Foldamers reveal and validate therapeutic targets associated with toxic α-synuclein self-assembly,” Nature Communications, vol. 13, article number 2272 (2022)). FIG. 4 shows an isolated view of the chemical structure of a portion of a variant of SK-129 shown on the right side of FIG. 3. Large arrow heads indicate locations on the OQ scaffolding structure at which selected side chains may be attached to tune the properties of the resulting ligand. In a model illustration, FIGS. 5A and 5B, showing a top view and a side view of the OQ scaffolding structure, illustrating the helical nature of the OQ scaffolding.

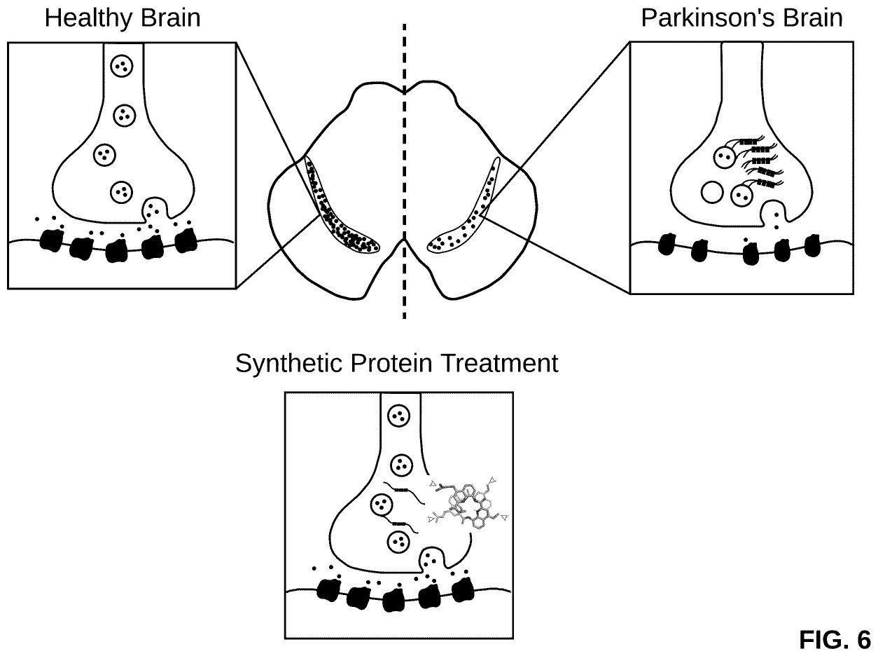

FIG. 6 illustrates the physiological mechanisms involved in the transmission of dopamine between neurons in a healthy brain and in a brain affected by Parkinson's Disease. As shown in the upper left inset, in a healthy brain, dopamine is readily transmitted between neuron endings to promote healthy dopaminergic neurons within the substantia nigra portion of the brain. However, in a brain affected by Parkinson's disease, as illustrated in the upper right inset of FIG. 6, the self-assembly of αS oligomers into non-functional amyloid structures prevents the transfer of dopamine, thus leading to loss of dopaminergic neurons and, consequently, progression of PD.

Referring to the bottom inset of FIG. 6, to inhibit the self-assembly of αS oligomers, a synthetic protein with a high affinity for αS oligomers may be introduced, thus resulting in the rescue of dopaminergic neurons. Further, the synthetic protein may be selected to mimic the conformation of the naturally occurring αS monomer to facilitate delivery of the synthetic protein through the blood-brain barrier and to the desired location of the brain. For instance, the synthetic protein illustrated in the bottom inset of FIG. 6 is the first reported foldamer with the ability to cross the BBB with high efficiency.

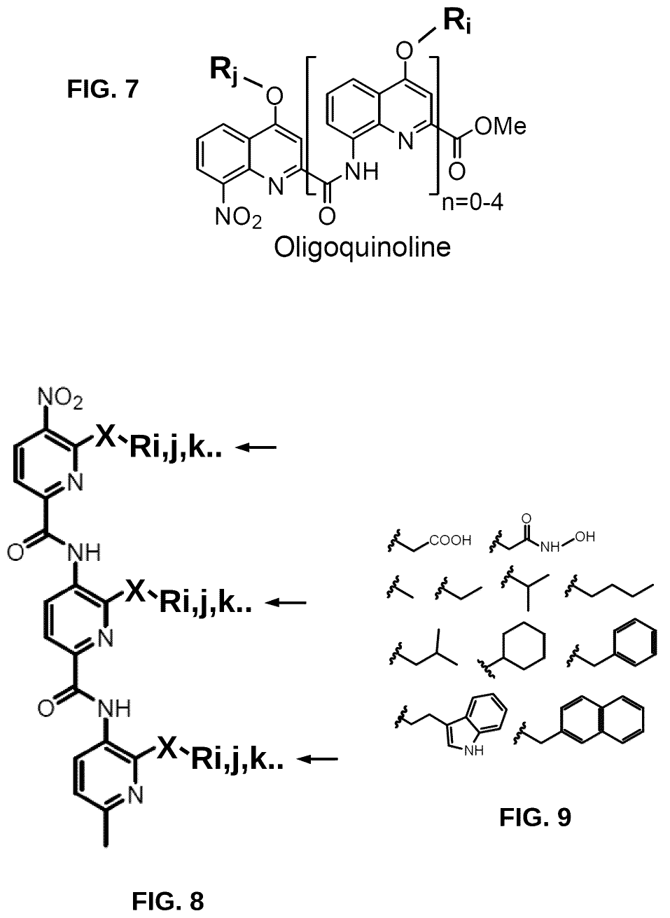

Further details of an oligoquinoline (OQ) structure are shown in FIG. 7, with a unit of OQ enclosed in brackets. The OQ structure may be used as scaffolding for the synthesis of foldamers with specific surface functionalities. As discussed above, multiple units of OQ may be joined to form a scaffolding structure with locations for attachment of specific side chains, as shown in FIG. 8. The scaffolding structure of FIG. 8 is shown with generic chemical structures Ri, Rj, Rk, etc. Some exemplary options for side chains attachable to the scaffolding structure of FIG. 8 are illustrated in FIG. 9. Synthesis of molecules with selected scaffolding and side chain structures may be performed using known protein synthesis protocols, such as illustrated in the attached Appendix A.

It is recognized herein that, more broadly, synthetic molecules that mimic the structure of certain proteins provide advantages, for instance, in increasing the affinity of such synthetic molecules to the target molecules so mimicked. For example, molecules that mimic the helical structure of certain proteins may be particularly suitable for therapeutic uses in inhibiting the self-assembly of proteins into non-functional amyloid structures as well as for delivery to the target therapeutic locations through the blood-brain barrier. Other features of target molecules that may be mimicked include, and are not limited to, molecule composition, length, curvature or spirality, and size and composition of the sidechains. For instance, the chemical diversity of the side chains of the selected scaffolding may be synthetically tuned for the optimization of their interactions with protein targets of interest. The scaffolding/backbone selected as a basis of the synthesized molecule may be optimized, for example, for antagonist activity toward target molecules.

As a specific example, the recognition that the helical structure of the alpha-Synuclein oligomers make them exhibit high affinity to synthetic foldamers, such as those based on OQ scaffolding may be valuable in selecting features of synthetic molecules for treating conditions associated with αS aggregation. That is, the helical nature of both alpha-Synuclein oligomers and the foldamer scaffolding is such that, with the appropriate sidechains selected and attached to the scaffolding, the resulting foldamer may be used as targeted therapeutics for crossing the blood brain barrier, thus preventing unwanted aggregation of amyloid proteins and slowing the progression of neurodegenerative diseases such as PD. Other synthetic protein mimetics are contemplated and are considered a part of the present disclosure.

The OQ scaffold approach can be applied to numerous disease models including Parkinson's disease, Alzheimer's disease, Lewy body dementia, multiple system atrophy, and diabetes, which are known to be caused by the aggregation of Synuclein proteins. These foldamer scaffolds are synthetically tunable meaning they can be engineered to specifically target a large variety of diseases that stem from aberrant protein-protein interactions. Further, the selection of the appropriate scaffolding structures and side chains enable targeting other proteins known to be associated with other diseases.

As an example, a selected foldamer (such as SK-129) based on the OQ scaffold may be specifically designed to interact with toxic αS oligomers to inhibit αS aggregation and the formation of αS fibers. For instance, whereas αS monomers occur naturally as native conformation in humans, it has been demonstrated in model studies that SK-α129 binds with a ten-fold higher affinity to pathological high molecular weight αS oligomers than αS monomers. In this way, a foldamer such as SK-129 may inhibit intracellular αS aggregation to promote rescue of degeneration of midbrain dopaminergic neurons, motility recovery, and improved behavioral deficits. Further, by selecting synthetic proteins such as SK-129 and variants that mimic the helical structure of αS monomers, such proteins may be used to as therapeutics that efficiently cross the blood-brain barrier. Additionally, by swapping out the side chains to form analogs of the foldamer, the interaction between the synthesized molecules with target molecules may be further tuned for therapeutic purposes.

More specifically, OQ scaffolding may be synthesized in a stepwise process via amide coupling of functionalized quinoline monomers to elongate the backbone. The OQ backbone presents chemically diverse sidechains capable of specific chemical interactions with mutant αS. For instance, SK129 presents carboxylic acid and isopropyl functional groups that interact with high specificity for the N-terminal lysine and hydrophobic amino acid residues of αS. Since these OQs are constructed through a series of amide coupling reactions, the foldamer scaffold can be synthetically tuned to increase its interaction with mutant αS to optimize the antagonist activity of OQs against αS aggregation. Various side chain compositions may be selected to synthesize analogs of the synthesized proteins to tune the interaction. For example, the use sulfonate and phosphonate sidechains in place of the carboxylic acid moiety and/or hydrophobic analogs of the isopropyl group will change the behavior of the synthesized molecules with respect to the target αS oligomers. In an example, OQs may be synthesized from a pool of functionalized monomers to enable rapid generations of analogs of SK129 to optimize its antagonist activity against a specific phenomenon, such as αS aggregation.

For instance, an OQ based library may be used to identify a potential candidate. In the present example, SK-129 may be selected as a potent antagonist of αS aggregation as it is known to specifically bind to αS oligomers (pathological conformation) with 10-fold higher affinity than αS monomers (native conformation) in cellular, neuronal, and C. elegans models and efficiently crosses the BBB without demonstrating any toxicity in a mouse model. Further, the OQ scaffolding structure is then synthetically tuned to further enhance their antagonist activity against αS aggregation and PD phenotypes.

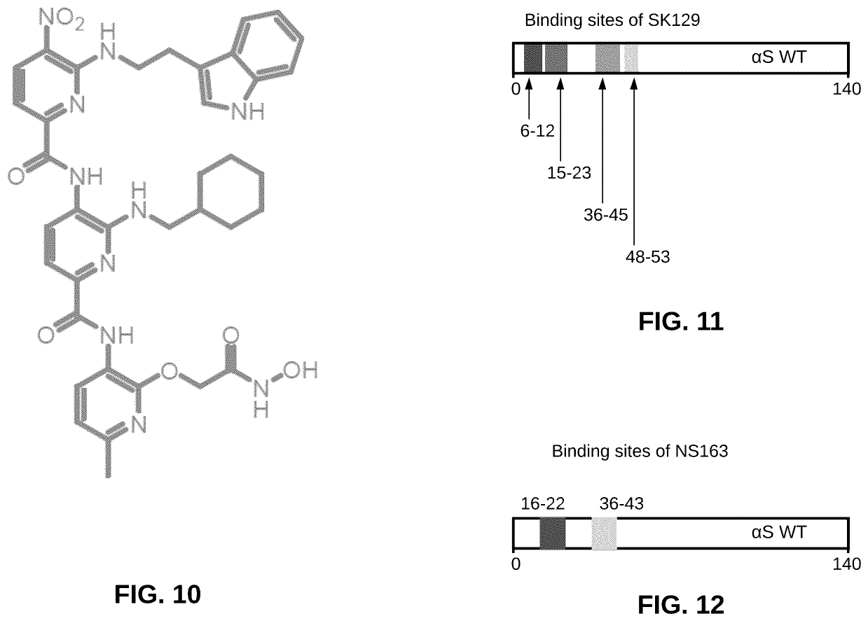

Further, other scaffolds, such as oligopyridylamides, may be tailored to target other proteins. For example, FIG. 10 shows a chemical diagram of NS-163, which may be another ligand that may target αS sequences. Whereas SK-129 may present greater tunability due to the greater number and variety of binding sites (see FIGS. 11 and 12), NS-163 may still be used for therapeutic purposes in specific contexts. In other words, oligopyridylamids may be an appropriate scaffolding approach for certain types of synthetic protein treatments. The oligoquinoline (foldamers) are one example of ligands that target the synuclein oligomers, and oligopyridylamides is another example of synthetic protein mimetic ligands that target the synuclein oligomers. These and other scaffolds that target the synuclein oligomers to inhibit aggregation and rescue toxicity, using the component selection and protein synthesis approach described herein.

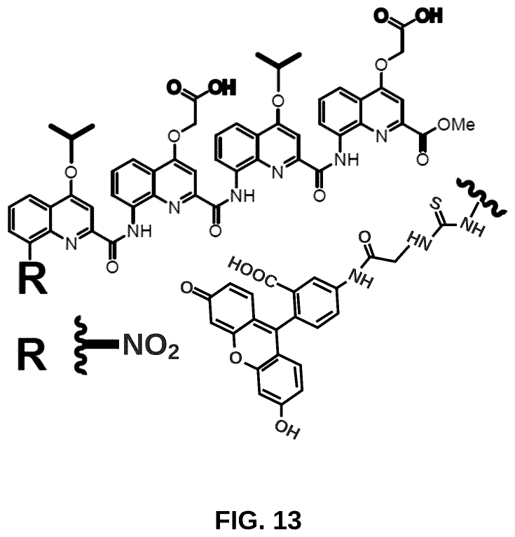

Similarly, an OQ-based molecule may be enhanced with a fluorescent probe. For example, SK-129 may also be used as a probe to identify high affinity ligands for αS oligomers and potent antagonists of αS aggregation from other existing libraries of ligands. For instance, a fluorescent analog of SK-129 (SK-129F as shown in FIG. 13) may be used as a probe that binds with high affinity to αS oligomers. In examples, a high-throughput fluorescence polarization displacement assay may be implemented to screen libraries of ligands against the SK-129-F- αS oligomers complex. The high affinity ligands for αS oligomers may be identified by detecting changes in the fluorescence polarization.

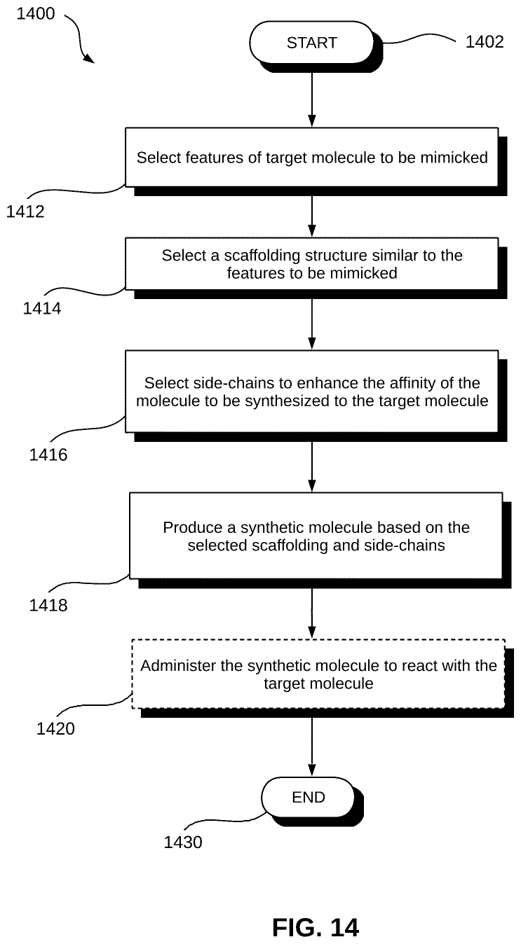

In embodiments, a method for using a synthetic protein molecule may include a process as illustrated in FIG. 14. As shown in FIG. 14, a process 1400 begins with a start step 1402 and proceeds to a step 1412 to select specific features of a target molecule (such as αS monomers) to be mimicked by the synthetic protein molecule. A scaffolding structure with characteristics similar to the selected specific features of the target molecule is selected in a step 1414. For instance, if the target molecule exhibits a helical structure, then a scaffolding structure also exhibiting a helical structure may be selected in step 1414.

Process 1400 then proceeds to a step 1416 to select specific side-chains to enhance the affinity of the synthesized molecule to the target molecule. The synthetic molecule, based on the selected scaffolding and side-chains, is produced in a step 1418. Optionally, the synthetic molecule so produced may be administered to a patient to react with the target molecule, and the process terminates in an end step 1430.

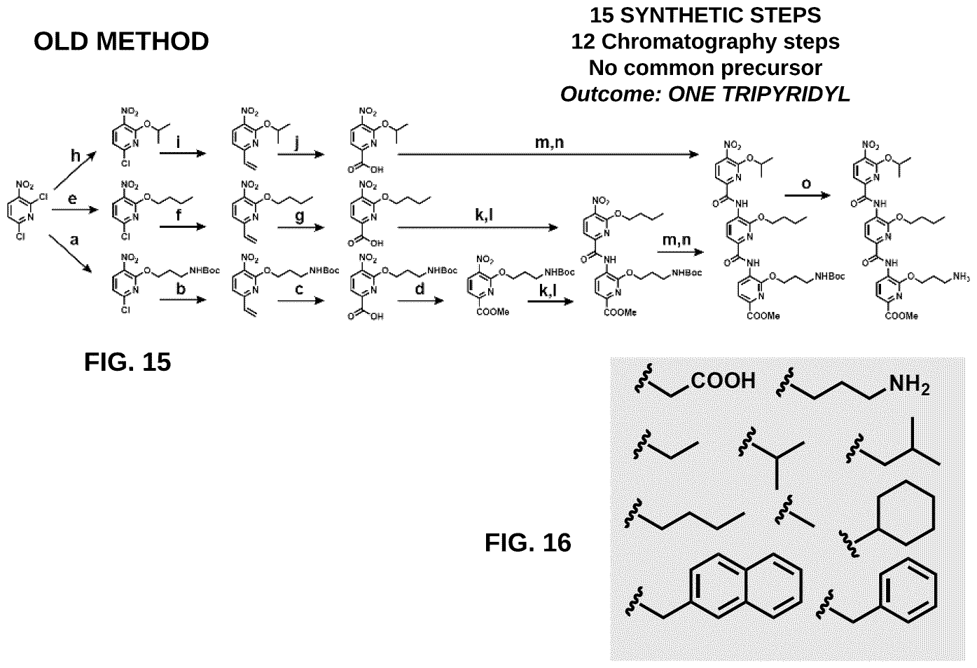

While oligopyridylamides mentioned above may provide a suitable synthetic protein mimetic ligand for targeting synuclein oligomers, it is cumbersome to customize the behavior of oligopyridylamides as existing methods to synthesize these molecules involve at least five synthetic steps and three chromatographic purifications per side-chain. An exemplary process is illustrated in FIG. 15, with the various side-chain options shown in FIG. 16. For instance, as shown in FIG. 15, starting with an alcohol molecule, the behavior of the monomer with different side-chains may be explored in parallel processes.

As one example, FIG. 15 shows parallel paths “a”, “e”, and “h” to synthesize pyridyl monomers including alkylation, oxidation, esterification, substitution, and coupling of 2,6-dichloro-3-nitropyridine. Each synthetic step must be evaluated with a chromatography step to characterize the molecule behavior with respect to the target molecule. In this particular example, without a common precursor, fifteen synthetic steps and twelve chromatography steps are required to produce one tripyridyl. While previous peptide-mimetic antagonists were identified after screening a library of molecules, without utilizing a systematic approach to increase their activity, such processes are extremely time consuming and cost prohibitive to even evaluate the use of the ten different side-chain options shown in FIG. 16.

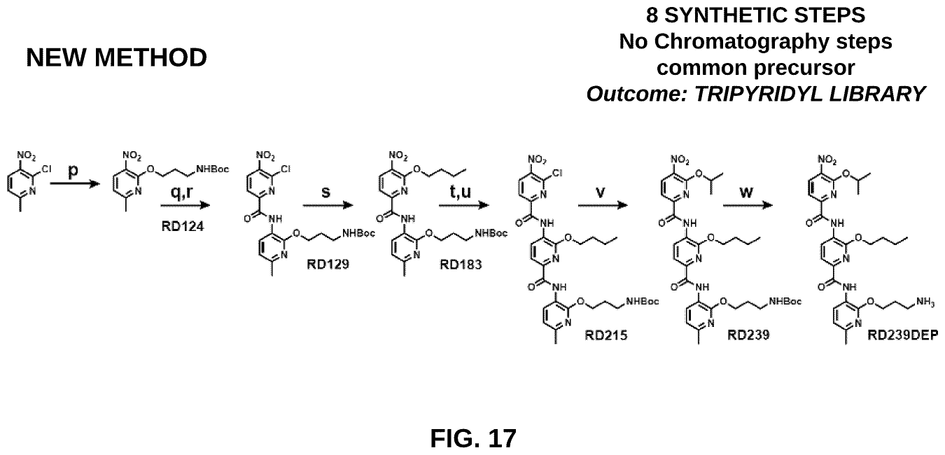

In contrast, an improved method described herein begins with a common precursor and requires no chromatography steps. For example, as shown in FIG. 17, the improved method begins with alcohol as the substitution reagent, then introduces side-chains to produce a library of tripyridyl variations in just eight synthetic steps, with no chromatography steps required. The improved method uses a combination of a fragment library approach and a structure-based approach to systematically screen and optimize the antagonist activity of synthetic protein mimetics against, for instance, aberrant protein-protein interactions such the aggregation of αs, a process associated with PD.

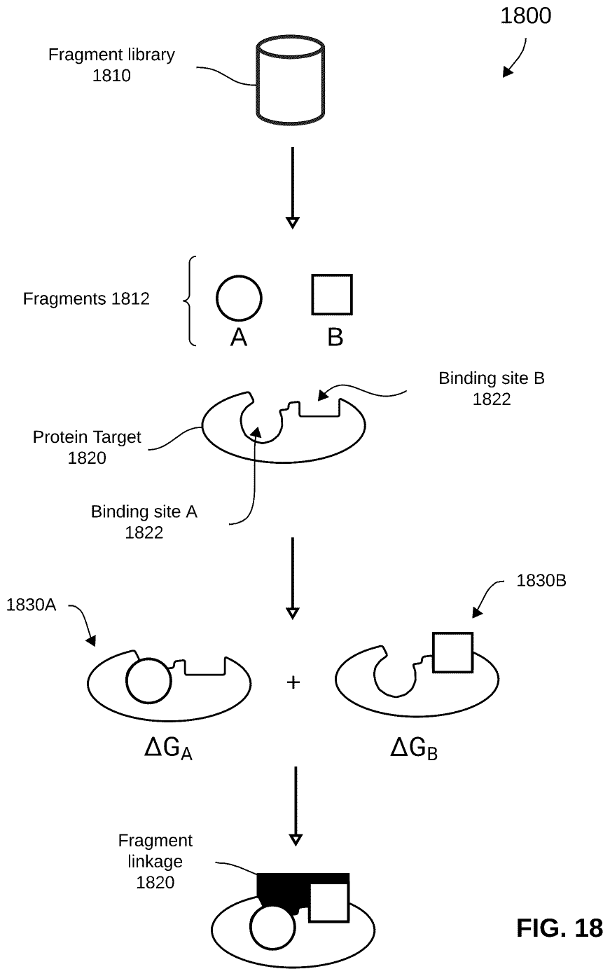

A typical fragment library approach is illustrated in FIG. 18. The fragment-based approach has emerged as a promising method for drug discovery. This approach includes screening a library (shown as fragment library 1810) of weakly binding, small molecule-based fragment candidates to select suitable fragments 1812 (shown as a circle A and a square B in FIG. 18) against a selected, protein target 1820. Fragments A and B bind at different locations (binding sites 1830A and 1830B, respectively) on protein target 1820 with weak to moderate affinities. Subsequently, the fragments are covalently linked (represented by a fragment linkage 1840) to generate high-affinity ligands for the target protein.

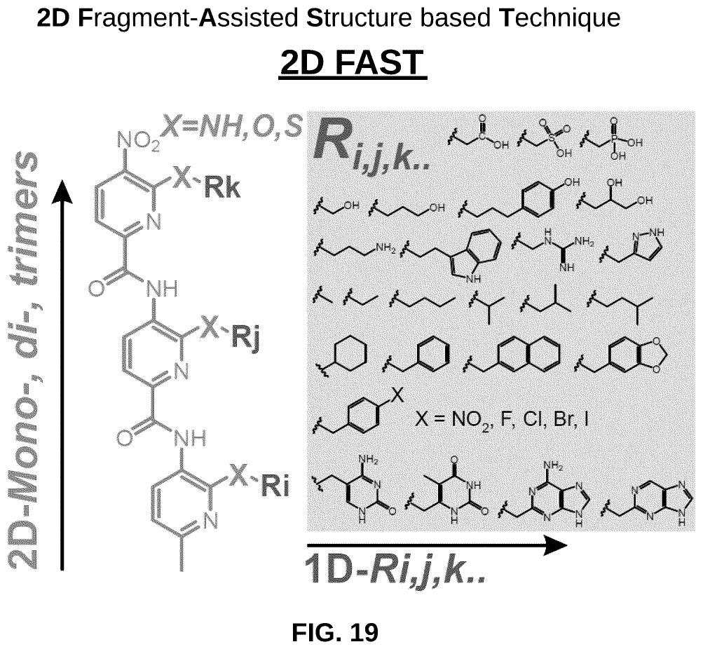

In contrast, a 2-dimensional Fragment-Assisted Structure-based Technique (2D FAST) method, as graphically summarized in FIG. 19, involves one-pot amide coupling reactions using acid chlorides, thus column chromatography is not required. In a first dimension, along the x-axis as shown in FIG. 19, the various side-chains of interest are considered. A variety of side-chains are introduced in the synthesis process for a specific monomer, dimer, or trimer, screened for desired characteristics with respect to target protein, then the best performing molecule is selected for further consideration. In a second dimension, along the y-axis as shown in FIG. 19, different protein quaternary structures are considered in the 2D FAST process. Through performing substitutions with primary amines and thiols, purification is only required for those side-chains that are nonvolatile or have a reduced solubility in water. By screening a library of dipyridyls, this approach implements a systematic optimization to increase activity and specificity for the protein target with a reduced number of synthetic procedures and chromatography requirement.

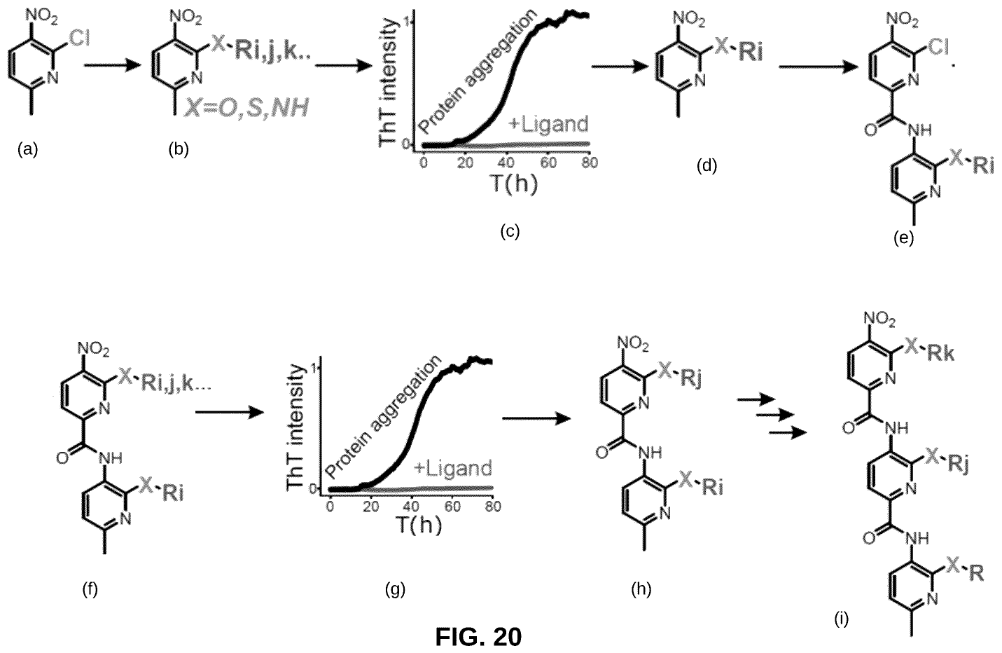

An exemplary process is shown in FIG. 20. As shown in FIG. 20, the 2D FAST process begins with an alcohol molecule (step (a)), then a variety of side-chain options (represented by X =O, S, NH with side chain options R_{i,j,k . . .} (step (b)) are added to the alcohol molecule to form a variety of monomers with the different side-chain options attached thereto. The synthesized monomers are evaluated in a Thioflavin T (ThT) dye-based aggregation assay to be screened against the aggregation of αS (step (c)), and the best performing monomer is selected in a step (d). The selected monomer is combined with a second molecule to form a dimer (step (e)), and various side-chain options are again considered for the second molecule (step (f)). The resulting dimers are again evaluated with a ThT assay (step (g)), and the best performing dimer is selected in a step (h). The process is repeated to screen for the best performing trimer (step (i)), and so on.

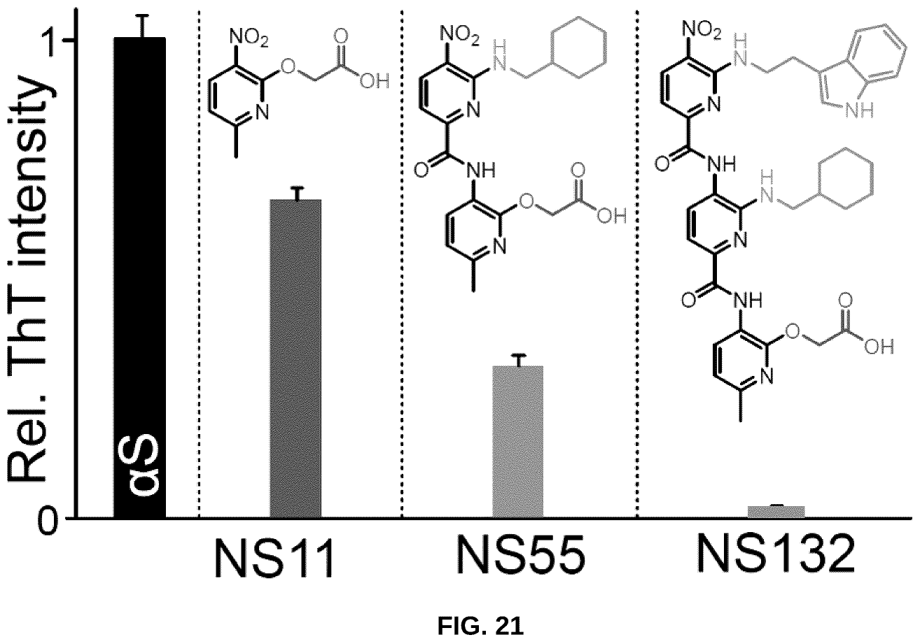

As a specific example, FIGS. 21-25 NS163, the molecule discussed above as a potent antagonist of α-Synuclein aggregation related to Parkinson's disease, was synthesized with just nine synthetic and three chromatographic steps total. In particular, FIG. 21 is a graph comparing the relative ThT intensities of various molecules synthesized using the 2D FAST process, in accordance with embodiments. The lower the ThT intensity, the higher the antagonist activity of ligands against the target protein (synuclein aggregation in this example). The data shown in FIG. 21 show that, as the fragments grow, the antagonist activity becomes higher.

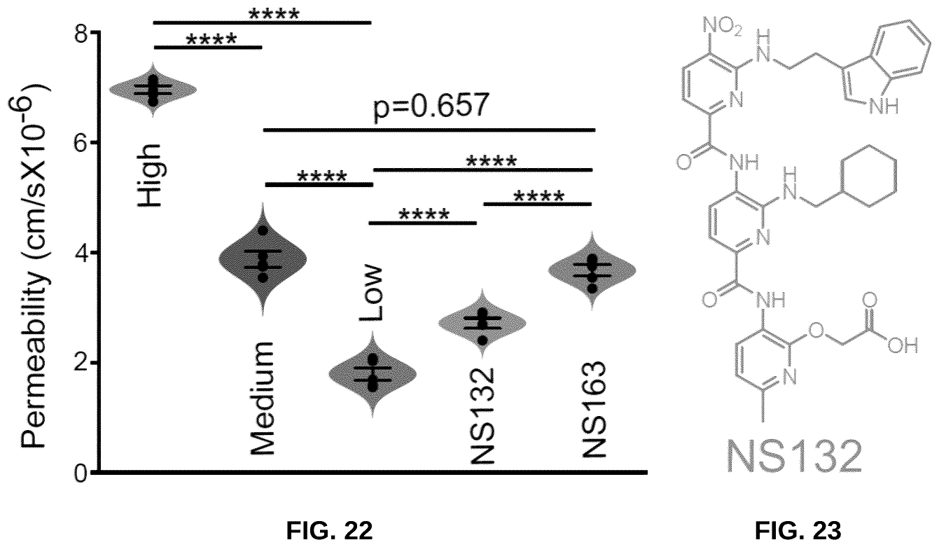

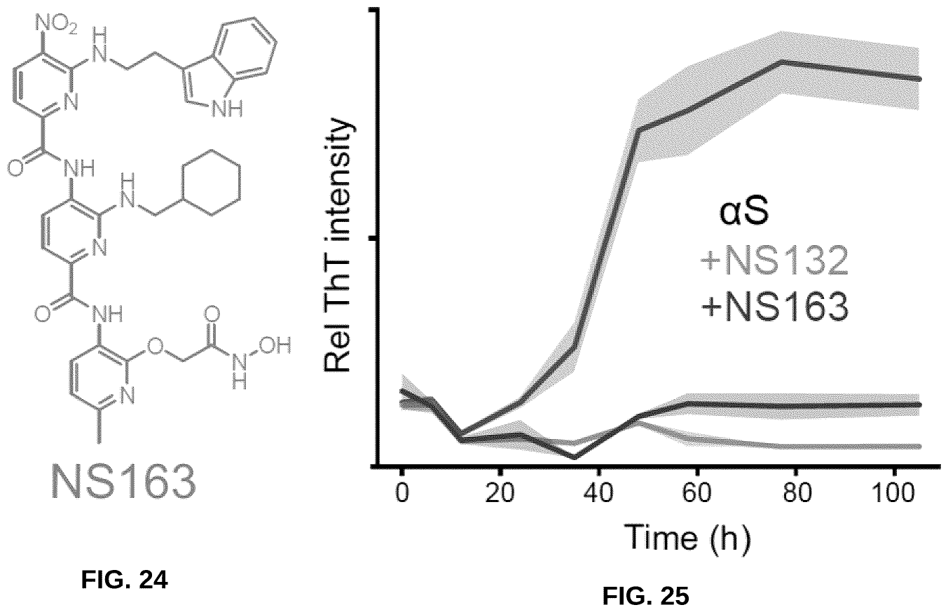

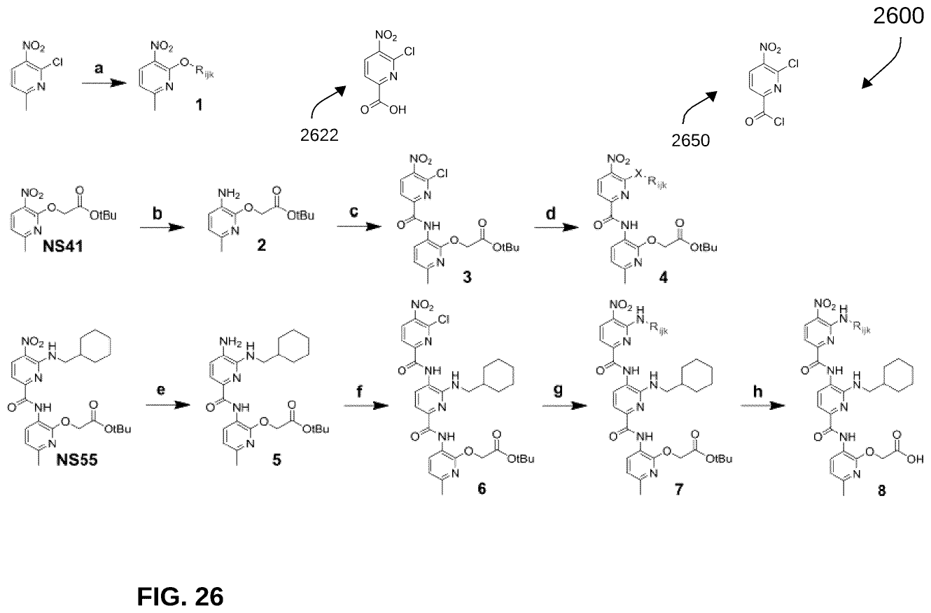

FIG. 22 is a graph comparing the cell permeability of various ligands identified and synthesized using the 2D FAST approach and assessed using the Parallel Artificial Membrane Permeability Assay (PAMPA) technique. FIG. 23 shows a chemical diagram of NS132, suitable for use with certain embodiments. FIG. 24 shows a chemical diagram of NS163, suitable for use with certain embodiments. FIG. 25 is a graph comparing the relative ThT intensities of various molecules synthesized using the 2D FAST process in the presence of αS, in accordance with embodiments.

By screening a library of dipyridyls, NS55 was identified as a potent inhibitor of α-Synuclein aggregation which was then systematically optimized at the tripyridyl stage. The most potent tripyridyl (NS132) was further enhanced for increased cell permeability and activity (NS163) within a C. elegans Parkinson's disease model. In fact, NS132 and NS 163 were shown to be very effective ligands in rescuing PD phenotypes in multiple C. elegans PD models, including degeneration of dopaminergic neurons, increase in reactive oxygen species, and reduced motility.

The methods described herein significantly reduces the number of labor-intensive synthetic procedures in the development of protein mimetics and involves a systematic optimization in the targeting of pathological protein species. In fact, the 2D FAST approach has the potential to be applied to other oligoamide scaffolds and beyond to identify potent and specific antagonists of various pathological protein-protein interactions associated with a plurality of diseases.

It is noted that, while the 2D FAST approach has been used to identify specific synthetic molecules that are effective for specific protein targets (e.g., alpha-Synuclein, A-beta, and tau, as described below in the Examples), specific details of the synthesis and analysis methods have not been described in detail. That is, while the 2D FAST approach had been generally referenced in past publications as an approach to synthesizing and evaluating a large variety of molecules in a condensed amount of time, past publications have only discussed the experimental performance of specific compounds that had been identified (e.g., those listed below with respect to Examples 1-3) and not the details of 2D FAST approach itself, until the disclosures presented in a publication involving the inventors of the present disclosure (see N.H. Stillman, “Protein mimetic 2D FAST rescues alpha Synuclein aggregation mediated early and post disease Parkinson's phenotypes,” Nature Communications, vol. 15:3658 (2024); https://doi.org/10.1038/s41467-024-47980-4). Whereas oligopyridylamides scaffolds were known, there was only a small library of known compatible side chains due to inherent limitations in the synthesis processes. The application of the 2D FAST approach has now expanded from 11 known side chains to 35 side chains to date, and may be further expanded due to the advantages provided by the 2D FAST approach. The 2D FAST approach may be further applied to other protein targets and synthesis methodologies.

### Example 1-αS Aggregation Inhibitors

### Synopsis

Aberrant protein-protein interactions (aPPIs) are associated with an array of pathological conditions, which make them important therapeutic targets. The aPPIs are mediated via specific chemical interactions that spread over a large and hydrophobic surface. Therefore, ligands that can complement the surface topography and chemical fingerprints could manipulate aPPIs.

Like oligoquinoline scaffolding discussed above with respect to Parkinson's Disease, oligopyridylamides (OPs) are synthetic protein mimetics that have been shown to manipulate aPPIs. However, the previous OP library used to disrupt these aPPIs was moderate in number (˜30 OPs) with very limited chemical diversity.

Using OPs as the basis, the 2D FAST approach was used to identify potent antagonists of α-Synuclein (αS) aggregation, a process central to Parkinson's disease (PD) as discussed above. Using fragment-based screening of large chemical space in OPs, the approach led to the identification of NS132 as an antagonist of the multiple facets of αS aggregation. Further, NS163 has been identified as an analog with better cell permeability without sacrificing activity. OPs have been shown to rescue αS aggregation mediated PD phenotypes in muscle cells and dopaminergic (DA) neurons in C. elegans models. Further, OPs have been demonstrated prevent the progression of PD phenotypes in a novel post-disease onset PD model. (See, for example, “A 2D Fragment-Assisted Protein Mimetic Approach to Rescue α-Synuclein Aggregation Mediated Early and Post-Disease Parkinson's Phenotypes,” bioRxiv preprint posted 2022 Jul. 13, doi: https://doi.org/10.1101/2022.07.11.499659)

Specific details of the synthesis of NS163 using the 2D FAST approach in this example are described below and illustrated in FIG. 26.

FIG. 26 illustrates a general synthesis process for synthesizing Oligopyridylmides (Ops) using the 2D FAST approach. The lettering of the steps corresponds to the processing steps as shown in FIG. 26.

### a. Synthesis of Starting Monomers

In general, a primary alcohol NaH (60% dispersion in mineral oil) or Na metal is mixed with toluene for 50 min at 0° C., then 5 hours at room temperature.

Then, to synthesize a starting monomer (1) in FIG. 26, a solution of 6-chloro-5-nitro-2-picoline (2.00 g, 11.6 mmol) in toluene (30 mL) under argon (gas) is stirred at 0° C. for 15 min before adding a primary alcohol [tert-butyl 2-hydroxyacetate for NS41] (18.5 mmol, 1.6 eq.). After stirring the solution for an additional 15 min at 0° C., NaH (60% dispersion in mineral oil) or Na metal (18.5 mmol, 1.6 eq.) is added incrementally over 20 min. The reaction is then stirred at 0° C. for 50 min and then at room temperature for 5 hours. After the disappearance of the starting material is confirmed using thin-layer chromatography (TLC), the reaction mixture is partitioned between ethyl acetate (EtOAc) and brine. The organic layers are combined, dried over anhydrous Na₂SO₄, and concentrated under vacuum, resulting in a yellow to brown solid, with 78.1-97.2% yield.

### b. Arylamide Reduction

To reduce arylamides and produce compound (2) in FIG. 26, the step begins with providing NS41. Palladium on activated carbon (Pd/C) (20% by wt., 0.298 g) is added to a solution of nitro arylamide (1.49 g, 5.55 mmol) in EtOAc (15 mL) and continuously stirred while bubbling H₂ (gas) for 3 hours at room temperature. After confirming the disappearance of the starting material by TLC, the reaction is filtered and concentrated. It is noted that the use of a rotary evaporator in this reaction is ill-advised due to the risk of product degradation. The product of this reaction (red/brown oil, 95.6%) is used in the next step without further characterization, in certain embodiments.

### b-1. Synthesis of 6-chloro-5-nitropicolinic Acid to be Used in Process Step c Below

To produce 6-chloro-5-nitropicolinic acid molecule 2622 in FIG. 26, to be used in producing compound (3), potassium dichromate (11.4 g, 38.6 mmol, 1.3 eq.) is added portion wise to a stirring solution of 6-chloro-5-nitro-2-picoline (5 g, 29.0 mmol) in concentrated H₂SO₄ (45 mL) over 20 min and refluxed at 60°° C. overnight. After confirming the absence of starting material via TLC, the reaction is placed on ice and quenched with water (25 mL). The reaction is then extracted with EtOAc (6×125 mL), dried over Na₂SO₄, and concentrated under vacuum to yield a pale yellow solid (5.63 g, 96.0% yield) as the pure product.

### c. One-Pot Amide Coupling (2630)

To produce compound (3) of FIG. 26, a solution of 6-chloro-5-nitropicolinic acid (compound 2622 in FIG. 26) (0.335 g, 1.65 mmol, 1 eq.) synthesized as described above, and compound (2) (0.394 g, 1.7 mmol) in dichloromethane (DCM) (anhydrous, 10 mL) is equilibrated for 15 min at 0°° C. under argon (gas). Triethylamine (TEA) (0.690 mL, 4.96 mmol, 3 eq.) is added to the stirring solution and equilibrated for an additional 60 sec at 0° C., followed by the addition of thionyl chloride (0.360 mL, 4.96 mmol, 3 eq.). The reaction mixture is stirred for 45 min at room temperature, and the disappearance of starting material is confirmed by TLC. The volatiles are removed on a rotary evaporator (rotovap), and the resulting product is partitioned between EtOAc and 1M HCl (1 ×15 mL). The organic layer is then washed with 1M NaOH (3×15 mL), followed by brine (1×15 mL), dried over anhydrous Na₂SO₄, and concentrated under vacuum, resulting in a yellow solid (0.699 g, 94.3% yield).

### d. Aromatic Substitution

To produce compound (4) of FIG. 26, a primary amine (cyclohexylmethanamine for NS55) (0.355 mmol, 10 eq.) and N,N-Diisopropylethylamine (DIPEA) (0.071 mmol, 20 eq.) are added to a solution of compound (3) (0.015 g, 0.036 mmol) in DCM (3 mL) and stirred at room temperature for 3 hours.

For reactions involving reagents that exhibit low solubility in DCM, e.g., tryptamine and C-(1-H_Pyrazol-3-yl)-methylamine, DMF may be substituted as the solvent.

The reaction mixture is dried following confirmation that the starting material had disappeared via TLC. For the removal of non-volatile reagents, flash chromatography may be required (10 to 80% EtOAc in hexanes, v/v, over 8 min). The products appear as yellow solids with a yield ranging from 48.0% to 94.3%.

### d-1. Synthesis of 6-chloro-5-nitropicolinoyl chloride to be Used in Step e. Below

To form 6-chloro-5-nitropicolinoyl chloride molecule 2650 of FIG. 26, a solution of 6-chloro-5-nitropicolinic acid (compound 2622 of FIG. 26 as described above) (0.500 g, 2.47 mmol) in SOCI₂ (10 mL) is refluxed overnight at 70°° C. After confirming the reaction is complete via proton nuclear magnetic resonance (1H NMR), the volatiles are removed under vacuum and the final product is preserved under argon (gas) at −20° C., resulting in a pale brown solid (0.516 g, 94.8% yield).

### e. Arylamide Reduction

To produce compound (5), a similar process as process step b described above may be used. The process begins by providing NS55. Pd/C (20% by wt., 0.298 g) is added to a solution of nitro arylamide (1.49 g, 5.55 mmol) in EtOAc (15 mL) and continuously stirred while bubbling H₂ (gas) for 3 hours at room temperature. After confirming the disappearance of the starting material via TLC, the reaction is filtered and concentrated. Again, the use of a rotovap in this reaction is ill-advised due to the risk of product degradation. The product of this reaction, a red/brown oil (95.6% yield) may be used in the next step without further characterization.

### f. Method for Tripyridyl Amide Coupling

To form molecule (6) of FIG. 26, a solution of 6-chloro-5-nitropicolinoyl chloride (0.076 g, 0.342 mmol, 1.3 eq) in dichloroethane (DCE) (10 mL) is stirred at 0° C. for 10 min. After equilibration, saturated NaHCO₃ (aqueous, 10 mL) ais added and the solution stirred vigorously for 1 minute.

A solution of compound (5) (0.124 g, 0.263 mmol) in DCE (3 mL) is then added dropwise over 60 sec and the solution is stirred at 0° C. for 10 minutes. Once TLC has confirmed the disappearance of the starting material, the resulting solution is partitioned between DCM and saturated NaOH. The combined organic layers are washed with brine, dried over Na₂SO₄, and concentrated under vacuum (orange solid, 0.168 g, 97.4% yield).

### g. Aromatic Substitution

To produce compound (7), a primary amine or thiol (tryptamine for NS132) (0.355 mmol, 10 eq.) and N,N-Diisopropylethylamine (DIPEA) (0.071 mmol, 20 eq.) are added to a solution of compound (6) (0.015 g, 0.036 mmol) in DCM (3 mL) and stirred at room temperature for 3 hours. For reactions involving reagents that exhibit low solubility in DCM, e.g., tryptamine and C-(1-H_Pyrazol-3-yl)-methylamine, DMF may be substituted as the solvent.

The reaction mixture is dried following confirmation that the starting material had disappeared via TLC. For the removal of non-volatile reagents, flash chromatography may be required (10 to 80% EtOAc in hexanes, v/v, over 8 min). The products appear as yellow solids with a yield ranging from 48.0% to 94.3%.

### h. Deprotection

To form compound (8) of FIG. 26, compound (7) (0.004-0.010 g) is dissolved in 3 mL of DCM followed by the addition of 1:1 Triethylsilane (TES): Trifluoroacetic acid (TFA) (0.5 mL each), stirring 60 sec prior to the addition of TFA. The solution is allowed to stir at room temperature for 3 hours, confirming the disappearance of starting material by TLC. Upon completion of the reaction, volatiles were removed under vacuum and the resulting product was washed with diethyl ether (3×2 mL, 0° C.) to yield the pure product, appearing as yellow-orange solids with a yield ranging from 50.8 to 97.1%.

Due to the sensitivity of indoles to acidic conditions, alternative methods of deprotection may be required for molecules containing this functional group. To a stirring solution of NS72 (0.012 g, 0.022 mmol) in acetonitrile (6 mL), water (40 μL) and elemental iodine (0.002 g, 30% mol) are added, and the solution is refluxed at 75 ° C. for 5.5 hours. After equilibrium is reached, the solution is extracted with Na₂S₂O₃ (aqueous, 4 mL) and DCM (2×10 mL), and the combined organic layers were dried over Na2SO4. Flash chromatography is used to recover the starting material (0 to 40% EtOAc in hexanes, v/v, over 6 min, 0.002 g, 16.4%) and to isolate the pure product (0 to 20% methanol in DCM, v/v over 6 min, 0.005 g 42.9% yield).

Next, to a stirring solution of NS132 (0.023 g, 0.030 mmol) in DCM (6 mL), zinc bromide (ZnBr₂, 0.033 g, 0.147 mmol, 5 eq) is added and stirred 72 hours at room temperature. Additional solvent may be added as needed to replace evaporated solvent. Subsequently, deionized water (30 mL) is added, and the solution stirred vigorously for an additional 2 hours. The solution is then extracted with DCM (3×10 mL) and the combined organic layers are washed with brine, dried over Na₂SO₄, and concentrated under vacuum (orange solid, 0.014 g, 65.2%).

### Synthesis of NS163

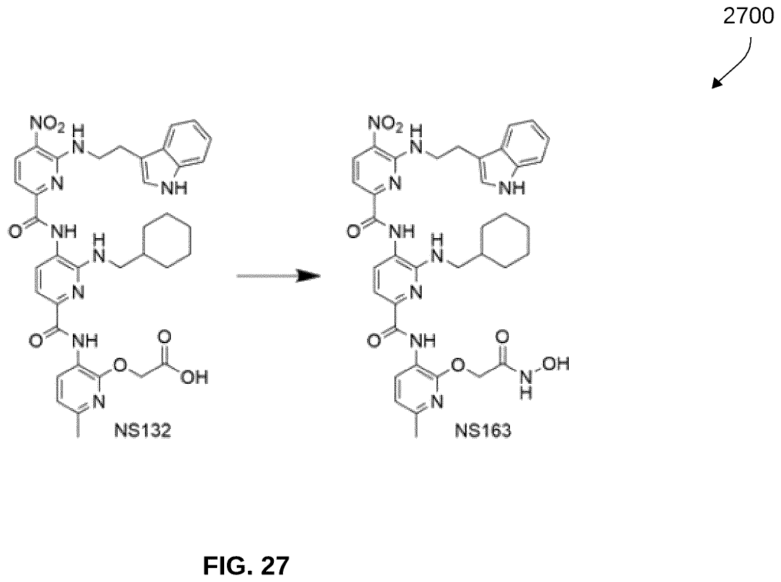

FIG. 27 illustrates formation of the NS163 molecule, as illustrated in FIG. 24. A solution of NS132 (0.009 g, 0.011 mmol) and CDI (0.003 g, 0.016 mmol, 1.5 eq.) in 2 mL of THF (anhydrous) is stirred at room temperature for 1 hour. After adding hydroxylamine hydrochloride (0.002 g, 0.022 mmol, 2 eq.), the reaction is stirred at room temperature for 20 hours. The volatiles are removed on a rotovap, and the resulting product is redissolved in EtOAc and washed with brine (3×5 mL). The organic layer is dried over Na₂SO₄ and concentrated under vacuum. Flash chromatography (0 to 15% EtOAc in Hexanes, v/v, over 15 min) yielded the final product (a yellow solid, 0.002 g, 23.7% yield).

Further details regarding the synthesis steps described in FIGS. 26 and 27 may be found in N.H. Stillman, et al., “Protein mimetic 2D FAST rescues alpha synuclein aggregation mediated early and post disease Parkinson's phenotypes,” Nature Communications, vol. 15, 3658 (2024); https://doi.org/10.1038/s41467-024-47980-4).

### Example 2-AβAggregation Inhibitors

The chromatography-free 2D FAST approach has been used to synthesize a highly diverse chemical library of OPs using the “common-precursor” approach as described above, thus significantly expanding the chemical diversity of OPs. In particular, an OP with identical chemical diversity to a pre-existing OP-base potent inhibitor of Aβ aggregation, a process central to Alzheimer's disease (AD) has been synthesized using this approach. The newly synthesized OP ligand (RD242) was proven to be very potent in inhibiting Aβ aggregation and rescuing AD phenotypes in an in vivo model. Moreover, RD242 was very effective in rescuing AD phenotypes in a post-disease onset AD model. (See specific details described in ““ Common-Precursor” Protein Mimetic Approach to Rescue AB Aggregation-Mediated Alzheimer's Phenotypes,” ACS Chem. Biol, Publication Date 2023 Jun. 27, https://doi.org/10.1021/acschembio.3c00120.)

### Example 3-2n4r Tau Aggregation Inhibitors

Another approach to combating neuropathic diseases such as AD is to target the tau protein. Tau is an intrinsically disordered protein, which is aggregation prone and implicated in numerous neurodegenerative diseases. Tau based proteinopathies, or tauopathies, are the leading cause of neurodegenerative diseases including AD and PD.

A library of small, protein-mimetic and foldamer molecules classified as oliopyridyls was synthesized using the 2D FAST approach and screened with recombinant 2N4R tau (hTau40 wt) for anti-aggregation and disaggregation activity. In particular, a library of protein-mimetic and foldamer oliopyridyl ligands (specifically an oliopyridyl trimer with interchangeable R groups) was designed and synthesized to interact with tau and α-synuclein based on charge and sequence, cell permeability, and aggregation attenuation. A number of these ligands reduced the aggregation of tau in biophysical assays, and further used in cellular and in vivo model systems.

As there exist a diverse set of distinct folds of tau for each tauopathy, three aggregation conditions resulting in three different fibril shapes were screened with the ligands and models. Specifically, no cofactor tau, ClearTau (consisting of heparin coated tubes to template aggregation of tau without heparin saturating the fibril), and tau in the presence of an excess of heparin were compared for aggregation propensity and aggregation end point for 25 μM tau be relative Thioflavin T (ThT) signal intensity. Transmission electron microscopy was used to confirm the aggregation of the samples.

After testing the library of molecules so synthesized, NS123, NS161, NS288, RD371, and RD373 identified as the ligands most effective in attenuating tau aggregation, with NS 161, RD371, and RD373 being most effective. Further experimental testing showed RD371 and RD373 ligands disrupted the aggregation of 2N4R tau rather than displacing ThT within a fibril to artificially reduce the ThT. Further, while NS161 prevented fibrils in the heparin aggregation condition to an extent, a few small fibrils remained. In contrast, in the presence of RD371 and RD373 tau remained more oligomeric and monomeric rather than aggregating, RD371 some tau +heparin fibrils formed while no cofactor tau fibrils formed, and RD373 prevented fibrils in all of the three conditions. The performance of the identified synthetic molecules have been confirmed in biophysical, cellular and C. elegans models (the experimental details are discussed in E. G. Oldani, et al., “Foldamers reveal and validate potential therapeutics associated with toxic 2N4R tau in multiple aggregation conditions,” unpublished).

To reiterate, unlike previous molecule synthesis approaches, the 2D FAST approach allows simultaneous assessment of multiple side chains via a fragment-based approach, enabling simultaneous screening and identification of the best affinity ligands against the protein target selected. The 2D FAST approach allows a much fewer number of synthetics as well as eliminating the need for chromatography steps. Further, the 2D FAST approach allows simultaneous screening and optimization of the ligands for a selected protein target. By using a common precursor to synthesize a large library of molecules, the 2D FAST approach enables the synthesis and evaluation of a much greater number of molecules in a much shorter amount of time.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”—whether explicitly discussed or not—and, conversely, were there only disclosure of the act of “protruding”, such a disclosure should be understood to encompass disclosure of a “protrusion”. Such changes and alternative terms are to be understood to be explicitly included in the description.

1. A method for synthesizing a synthetic molecule, the method comprising:
selecting a target protein;
selecting a common precursor;
providing at least first and second side-chains compatible with the common precursor;
forming a first monomer, the first monomer including the common precursor with the first side-chain attached thereto;
forming a second monomer, including the common precursor with the second side-chain attached thereto;
evaluating the first and second monomers using an aggregation assay in presence of the target protein to generate first results; and
selecting one of the first and second monomers as a selected monomer based at least in part the first results,
wherein the aggregation assay excludes chromatography steps.

2. The method of claim 1, further comprising:
providing at least third and fourth monomers compatible with the selected monomer;
forming a first dimer, including the selected monomer with the third monomer attached thereto;
forming a second dimer, including the selected monomer with the fourth monomer attached thereto;
evaluating the first and second dimers with the aggregation assay to generate second results; and
selecting one of the first and second dimers as a selected dimer based at least in part on the second results.

3. The method of claim 2, further comprising:
providing at least fifth and sixth monomers compatible with the selected dimer;
forming a first trimer, including the selected dimer with the fifth monomer attached thereto;
forming a second trimer, including the selected dimer with the sixth monomer attached thereto;
evaluating the first and second trimers with the aggregation assay to generate third results; and
selecting one of the first and second trimers as a selected trimer based at least in part on the third results.
